(19) United States
(12) Patent Application Publication
(10) Pub. No.: US 2025/0148125 A1
(43) Pub. Date: May 8, 2025

(54) CONTROLLED DATA ACCESS FOR DECENTRALIZED CLINICAL TRIAL SPACE

(71) Applicant: Pharmaco Investments, Inc., Wilmington, NC (US)

(72) Inventors: Melinda Bolling, Dry Ridge, KY (US); Chris Clendening, Cleves, OH (US); Viral Trivedi, Cincinnati, OH (US); Benjamin Eirich, Dry Ridge, KY (US)

(21) Appl. No.: 18/936,583

(22) Filed: Nov. 4, 2024

Related U.S. Application Data
(60) Provisional application No. 63/547,219, filed on Nov. 3, 2023.

(51) Int. Cl. G06F 21/62; G16H 10/20; G16H 10/60

(52) U.S. Cl. CPC G06F 21/6245; G16H 10/20; G16H 10/60

(57) ABSTRACT

Disclosed herein is a system and method for controlling data access within a decentralized clinical trial space. In various embodiments of the present invention, a patient portal system may be interconnected with a clinical trial database, a patient device configured to access a patient portal application associated with the patient portal system, and an intermediary system having an intermediary database. Data transmitted from the patient device to the patient portal system may comprise obfuscated patient data. Such obfuscated patient data may be transmitted to the intermediary database which may be configured to access the identifying patient data from the obfuscated patient data through a private key stored therein. The patient portal system may enable a patient to perform a variety of functions relating to a clinical trial, such as ordering a sample collection kit, to be fulfilled by a supply system interconnected with the intermediary database.

### CROSS-REFERENCE REFERENCE TO RELATED APPLICATIONS

The present Non-Provisional application claims priority to the earlier-filed and currently pending Provisional Application No. 63/547,219 having a filing date of Nov. 3, 2023, the contents of which are hereby incorporated by reference in its entirety.

### FIELD

This application generally relates to systems and methods for controlling data access in a decentralized clinical trial space, such as, for example, providing trial participants supply kit delivery and sample collection to and from their residence.

### SUMMARY

Clinical trials are an important part of research for new tests and treatments and are often subject to regulatory requirements. Traditionally, a participant in a clinical trial (also referred to here as a “patient”) visits a brick-and-mortar location to register for a trial and have one or more samples collected over one visit or multiple visits. As more and more systems and services move to online or remote options, providing a decentralized trial space has become an important consideration in the clinical trial industry. However, existing technologies for online ordering and fulfillment and pick-up fail to accommodate the unique workflows and data access controls associated with clinical trials. For example, clinical trials are often associated with regulatory requirements on anonymity and, thus, clinical trial providers are often restricted from having access to identifying information regarding patients, which includes names, email addresses, and physical mailing addresses, all of which are routinely collected and used in existing online ordering and pick-up technology. In other words, anonymity regulations and/or blinding requirements may necessitate the segmentation of data to promote compliance and trial efficacy.

However, due to these anonymity and/or blinding requirements, patients in a decentralized clinical trial environment often have no visibility on supply kit shipments shipped to their home as their clinical trial providers are unable to store and access the pertinent shipment data associated with the identifying information. In other words, because the data cannot be unblinded in conventional systems, patients using such systems must manually find the site and/or investigator contact details and call to obtain the status of their shipments and pickups. Naturally, the complicated nature of this process tends to result in lower compliance metrics for patients in clinical trials.

Accordingly, embodiments described herein provide systems and methods for addressing these and other technological problems by controlling access to data within an online ordering system and, in particular, an online ordering system associated with a decentralized clinical trial. As described in more detail below, the methods and systems described herein allow clinical trial providers and participants to manage supply kits provided to patients, manage patient and sample level data collected for a visit, and manage sample pick up and routing to a lab in a controlled manner to ensure proper access and separation of data in compliance with the anonymity requires discussed heretofore. The methods and systems described herein may also efficiently connect participants within a given clinical trial with their applicable trial sites, investigators, helpdesks, and any necessary third-party applications associated therewith. For example, some embodiments described herein enable a participant to launch a third-party application within an application installed on the participant's device (e.g., mobile phone, tablet, or the like), such as, for example, using single sign-on (SSO) technology or application programing interface (API) integration.

In view of the above, an exemplary method for protecting personal patient information in a decentralized clinical trial may comprise: (a) registering a patient portal application on at least one user device via a registration code, the registration code comprising a shared key configured to generate obfuscated patient data for transmittal to a patient portal system via at least one server, the patient portal system communicatively configured in connection with an intermediary system having an intermediary database and a clinical trial database; (b) receiving, at the user device from the server, a sample collection information, the sample collection information including an identifier of a supply kit available for order by at least one patient participating in the clinical trial; (c) providing, at the user device, the sample collection information within a user interface of the patient portal application; (d) receiving, at the patient portal system, the obfuscated patient data relating to the provided sample collection information from the user device; (e) transmitting the obfuscated patient data from the patient portal system to the intermediary database, the intermediary database comprising a private key configured to transform the obfuscated patient data into identifying patient data; and (f) transmitting the identifying patient data from the intermediary database to at least one supply system, the at least one supply system.

In at least one alternative embodiment, a method for protecting personal patient information in a decentralized clinical trial may comprise: (a) placing a patient portal system in communicative configured with a clinical trial database, an intermediary database, and at least one user device through at least one server; (b) storing, on the clinical trial database, key-coded data relating to at least one patient; (c) registering the at least one user device with the patient portal system through a patient portal application disposed thereon, such registration effectuated by at least one registration code provided to the at least one patient; (d) receiving, by the patient portal application, data relating to the at least one patient and generating obfuscated patient data therefrom according to an encoding algorithm; (e) transmitting the obfuscated patient data through the patient portal system and to the intermediary database, the intermediary database having at least one private key stored therein, the at least one private key configured to access identifying patient data from the obfuscated patient data; and (f) transmitting the obfuscated patient data from the intermediary database to at least one supply system communicatively configured in connection therewith.

Likewise, in at least one alternative embodiment, a system for controlling data access a patient's personal information in a decentralized clinical trial may comprise: (a) a patient portal system communicatively configured in connection with at least one patient device, a clinical trial database, and at least one intermediary database; (b) the at least one patient device comprising a processor communicatively configured in connection with a memory to provide a patient in input/output communication with the patient portal system through a patient portal application stored thereon; (c) the at least one patient device configured to scan at least one registration code, the at least one registration code configured to enable access by the at least one patient device to the patient portal application for the input of identifying patient data therein; (d) the registration code configured to apply an encoding algorithm via a shared key to the identifying patient data, the encoding algorithm configured to transform the identifying patient data into obfuscated patient data and key coded data; (e) the patient portal system configured to receive the obfuscated patient data and the key coded data from the at least one patient device through the patient portal application; (f) the patient portal system configured to provide the key-coded data to the clinical trial database; (g) the patient portal system configured to provide the obfuscated patient data to the intermediary database; (h) the intermediary database configured to apply a private key stored therein to the obfuscated patient data to access the identifying patient data therefrom; and (i) the intermediary database configured to provide the identifying patient data to a supply system communicatively configured in connection therewith.

### BRIEF DESCRIPTION OF THE DRAWINGS

Embodiments will be readily understood by the following detailed description in conjunction with the accompanying drawings. To facilitate this description, like reference numerals designate like structural elements. Embodiments are illustrated by way of example, not by way of limitation, in the figures of the accompanying drawings.

FIG. 1A is a block diagram illustrating a decentralized clinical trial system, in accordance with various embodiments.

FIG. 1B is a block diagram illustrating an encoding algorithm to be used in the decentralized clinical trial system of FIG. 1A, in accordance with various embodiments.

FIG. 2 is a block diagram illustrating a patient device included in the system of FIG. 1A, in accordance with various embodiments.

FIG. 3 is a block diagram illustrating a patient portal system included in the system of FIG. 1A, in accordance with various embodiments.

FIG. 4 is a flowchart illustrating a patient portal application workflow method, in accordance with various embodiments.

FIG. 5 is a flowchart illustrating a patient portal registration method, in accordance with various embodiments.

FIG. 6 is a flowchart illustrating a supply order method, in accordance with various embodiments.

FIG. 7 is a flowchart illustrating a sample kit pickup method, in accordance with various embodiments.

FIG. 8 is a flowchart illustrating an electronic requisition method, in accordance with various embodiments.

FIGS. 9A-9P depict a plurality of graphical user interfaces provided via a patient portal application to the patient, in accordance with various embodiments.

FIG. 10 is a block diagram illustrating a computing device implementing the patient portal system of FIG. 7, in accordance with various embodiments.

### DETAILED DESCRIPTION

Disclosed herein are systems, as well as related methods, computing and storage devices, and computer-readable media, for providing a decentralized clinical trial environment. The systems, methods, computing and storage devices, and computer-readable media disclosed herein may achieve improved performance relative to conventional approaches and, for example, may achieve improved performance over existing online ordering technology. As noted above, existing online ordering technology is inadequate for clinical trial settings or other settings where data access needs to be controlled such that particular systems and devices do not have access to particular data types, such as, for example, participant identifying information including, without limitation, a participant's name, email address, and/or physical mailing address.

The above-indicated problems in the state of the art can beneficially be addressed using various examples, aspects, features, and embodiments of systems and methods for data access control disclosed herein. Accordingly, the technological problems created by introducing clinical trials to a remote or online environment may be resolved by the particular computing systems, devices, and distribution of functionality and data described in the various embodiments herein. Thus, embodiments disclosed herein provide improvements to online ordering technology (e.g., improvements in the computer technology supporting such ordering functionality, among other improvements), such as anonymity-based data control solutions between various devices, systems, and databases associated with a decentralized clinical trial system.

In the following detailed description, reference is made to the accompanying drawings that form a part hereof wherein like numerals designate like parts throughout, and in which is shown, by way of illustration, embodiments that may be practiced. It is to be understood that other embodiments may be utilized, and structural or logical changes may be made, without departing from the scope of the present disclosure. Therefore, the following detailed description is not to be taken in a limiting sense.

Various operations may be described as multiple discrete actions or operations in turn, in a manner that is most helpful in understanding the subject matter disclosed herein. However, the order of description should not be construed as to imply that these operations are necessarily order dependent. In particular, such operations may not be performed in the order of presentation. Operations described may be performed in a different order from the described embodiment. Various additional operations may be performed, and/or described operations may be omitted in additional embodiments.

For the purposes of the present disclosure, the phrases “A and/or B” and “A or B” mean (A), (B), or (A and B). For the purposes of the present disclosure, the phrases “A, B, and/or C” and “A, B, or C” mean (A), (B), (C), (A and B), (A and C), (B and C), or (A, B, and C). Although some elements may be referred to in the singular (e.g., “a processing device”), any appropriate elements may be represented by multiple instances of that element, and vice versa. For example, a set of operations described as performed by a processing device may be implemented with different ones of the operations performed by different processing devices.

The description uses the phrases “an embodiment,” “various embodiments,” and “some embodiments,” each of which may refer to one or more of the same or different embodiments. Furthermore, the terms “comprising,” “including,” “having,” and the like, as used with respect to embodiments of the present disclosure, are synonymous. When used to describe a range of values, the phrase “between X and Y” represents a range that includes X and Y. As used herein, an “apparatus” may refer to any individual device, collection of devices, part of a device, or collections of parts of devices. The drawings are not necessarily to scale.

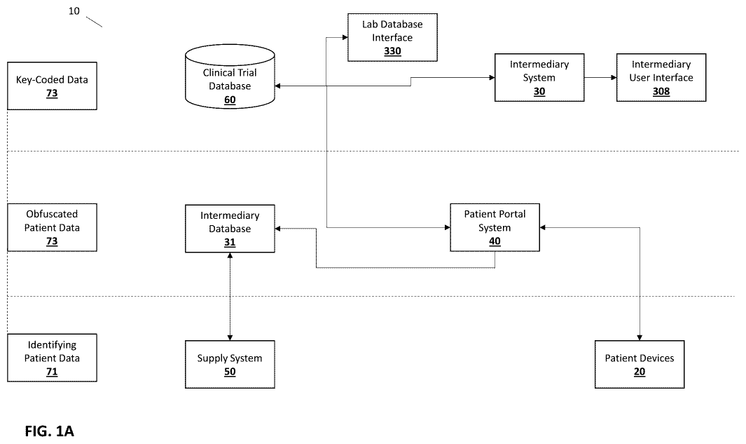

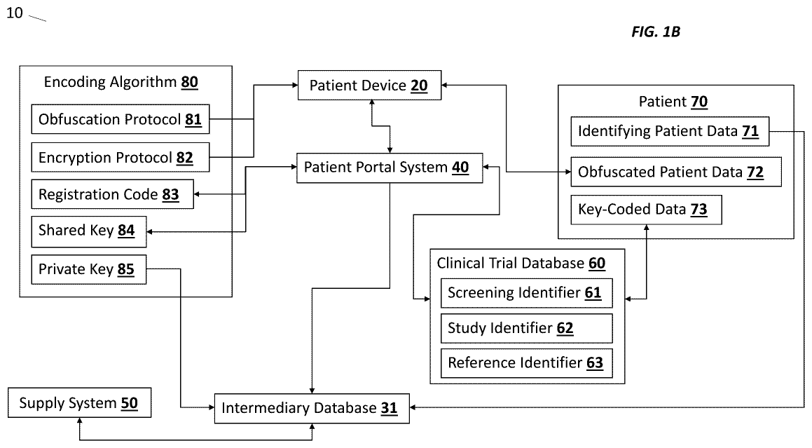

FIG. 1A is a block diagram illustrating a decentralized clinical trial (“DCT”) system 10, in accordance with various embodiments of the present disclosure. The DCT system 10 includes one or more patient devices 20, such as those user devices 304 operated by a patient and/or trial participant (alternative user devices 304 operated by others associated with a clinical trial are envisioned and discussed in greater detail hereafter), an intermediary system 30 having an intermediary database 31 and an intermediary user interface 308, a patient portal system (“PPS”) 40, and a supply system 50 (also referred to herein as a supply depot). As illustrated in FIG. 1A, the PPS 40 may further communicate with or have access to a clinical trial database 60, which may comprise or otherwise be interconnected with a lab database interface 330. In some embodiments, the clinical trial database 60 may be included as part of the PPS 40. Similarly, in some embodiments, the clinical trial database 60 may be distributed over multiple databases and/or systems, whether internal or external to the PPS 40.

The various components of the DCT system 10 may communicate over one or more communication networks or connections that may be implemented using wired communication components, wireless communication components, or a combination thereof and may include various types of networks or interconnections, such as, for example, a cellular network, a wide area network (such as, for example, the Internet), a local area network (such as, for example, a Wi-Fi® network), a short-range wireless network or connection (such as, for example, a Bluetooth® connection or near-field connection), or a combination of the foregoing. It may be understood, in some embodiments, one or more dedicated connections or communication channels may be used between one or more of the components of the system 10.

For ease of description, the system 10 illustrated in FIG. 1A includes a single patient device 20. However, it should be understood that multiple participants using multiple different patient devices 20 may communicate with and access the functionality described herein with respect to the system 10. Also, in some embodiments, the functionality described herein as being performed via the PPS 40 may be distributed over a plurality of devices, such as, for example, functionality described herein may be distributed between the PPS 40 and the clinical trial database 60, distributed between multiple computing devices included in a cloud computing or other distributed environment, or a combination thereof. Also, in some embodiments, the PPS 40 performs functionality in addition to the functionality described herein.

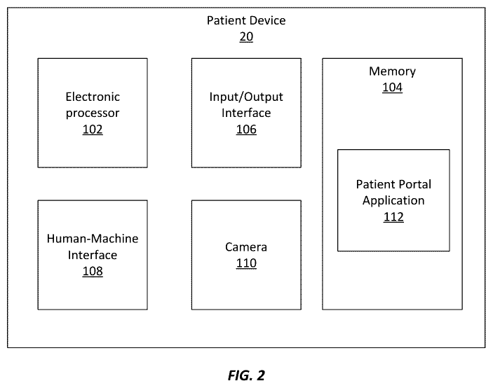

The patient device 20, such as the one depicted in FIG. 2, may comprise a computing device owned and/or operated by a patient 70 in a clinical trial, such as, for example, a smart phone, a tablet computer, a laptop computer, a smart wearable device, or the like. The patient device 20 may include a plurality of electrical and electronic components that provide power, operational control, and protection to the components and modules within the patient device 20. For example, as illustrated in FIG. 2, such a patient device 20 may include an electronic processor 102 (for example, an electronic microprocessor, microcontroller, or similar device), a memory 104 (for example, non-transitory, computer-readable memory), and an input/output (I/O) interface 106. The patient device 20 also includes one or more human-machine interfaces 108, such as, for example, a touchscreen, a display, a keypad or keyboard, a cursor-controlled device, a speaker, or a combination thereof. The human-machine interface(s) 108 receive data from the patient 70 and provide data to the patient 70, such as, for example, in audible form, textual form, graphical form, or a combination thereof. Such a patient device 20 may include a camera 110, which, as described in more detail below, allows the patient 70 to scan one or more codes. The patient device 20 may include additional or alternative components, including additional electronic processors and memory, or application specific integrated circuits (ASICs), as well as one or more input devices, output devices, or a combination thereof.

The components of the patient device 20 may be connected in various ways including, for example, a local bus and/or an internal bus configured to connect the various internal components thereof such as the electronic processor 102 and memory 104 to a motherboard. The electronic processor 102 is communicatively coupled to the memory 104 and executes instructions stored on the memory 104. The electronic processor 102 is configured to retrieve from the memory 104 and execute, among other things, instructions related to the control processes and methods described herein. For example, the memory 104 may include a program storage area and a data storage area, and the electronic processor 102 is connected to the memory 104 and executes computer readable instructions (“software”) stored in a random-access memory (RAM) of the memory 104, a read only memory (ROM) of the memory, or another non-transitory computer readable medium. For example, as illustrated in FIG. 2, the memory 104 may store a patient portal application 112 that, when executed by the electronic processor 102, communicates with the PPS 40 and, in some embodiments, the intermediary system 30 to access data and initiate communications within the system 10. The patient portal application 112 may include firmware, one or more applications, program data, filters, rules, one or more program modules, other executable instructions, or a combination thereof and, in some embodiments, the patient portal application 112, when executed, is configured to perform additional functionality than the functionality described herein. Also, as noted above, functionality described herein as being performed via execution of the patient portal application 112 may be distributed among multiple devices in the same or separate housing.

The input/output interface 106 of the user device 102 may be configured to transmit data to and receive data from one or more devices, networks, or systems external to the patient device 20. For example, as illustrated in FIG. 1, the patient device 20 (through the input/output interface 106) is configured to communicate with the PPS 40 and the intermediary system 30 over one or more communication networks or connections. It should be understood that the patient device 20 may communicate with the PPS 40, the intermediary system 30, or both through one or more intermediary devices (not shown).

As depicted in FIGS. 1A and 1B, data received from and/or provided to the patient device 20 may comprise identifying patient data 71, which includes any data which could be used to identify the patient such as a name, address, birth date, and billing information. In at least one embodiment, such identifying patient data 71 may only comprise such data only reasonably necessary to ship, for example, a supply kit, to the patient, including, without limitation, the patient's name and address. Likewise, the patient device 20 may additionally receive and/or provide obfuscated patient data 72, which may include data related to the patient such as confidential or sensitive data, but which is obfuscated, encrypted, or otherwise disguised in such a manner that the same may not be associated with the patient. In at least one embodiment, such obfuscated patient data 72 may be disposed in a non-human readable format, whether comprising a machine-readable format or otherwise. As may be understood, such obfuscated patient data 72 may, in certain instances, include the identifying patient data 71, albeit in an obfuscated form such that the same is not readable by one or more systems, such as the PPS 40. In at least one embodiment, such patient device 20 may further receive and/or provide key-coded data 73, which will be discussed in greater detail hereafter. In at least one embodiment, the patient device 20 is configured so that the same does not directly store either such identifying patient data 71 or such obfuscated patient data 72 within the memory 104 thereof.

Accordingly, it may be understood that, as the patient enters their data into the PPS 40 from the patient device 20, such data may be turned into such obfuscated patient data 72 via an encoding algorithm 80 before entering PPS 40. Such an encoding algorithm 80 may, in at least one embodiment such as the one depicted in FIG. 1B, be formed of two components: (a) an obfuscation protocol 81, which may perform one or more obfuscation techniques to the patient data; and (b) an encryption protocol 82, which may apply one or more encryption techniques to the patient data. For instance, in at least one embodiment of the present invention, the patient device 20 may be registered with the PPS 40 via a registration code 83, which may comprise, for instance, a quick response (“QR”) code. Such a registration code 83 itself comprise a shared key 84, which may be used to apply the aforementioned encoding algorithm to the patient's data. The generation and use of the registration code 83 will be discussed in greater detail hereafter.

In so doing, the encoding algorithm 80 may enable appropriate blinding of the patient's data between the various systems of the decentralized clinical trial system 10. For instance, once the obfuscation protocol 81 and encryption protocol 82 are applied to the patient device 20—i.e., after the registration of the patient device 20 with the PPS 40 via a registration code 83 having a shared key 84—the shared key 84 of the registration code 83 may be utilized by the above-mentioned protocols to generate the obfuscated patient data 72. Meanwhile, the private key 85 associated with such shared key 84 may be stored solely within the intermediary database 31. When the patient device 20 is used by the patient 70 to perform various functions—e.g., to order a sample kit, which may be based on the key-coded data 73 passed to the patient device 20 through the PPS 40, wherein such key-coded data 73 may comprise a screening identifier 61, as discussed hereafter—the patient device 20 passes the obfuscated patient data 72 to the PPS 40, where it is subsequently transmitted to the intermediary database 31. There, the private key 85 may be applied to the obfuscated patient data 72 to determine the identifying patient data 71. Such identifying patient data 71 may then be transmitted to the supply system 50 for the fulfillment of the patient's 70 requested action(s). As such, the PPS 40 may enable a patient 70 to fulfill their requests, and have visibility relating to the same, without receiving, storing, or accessing the identifying patient data 71.

As illustrated in FIG. 1A, and as previously discussed heretofore, the PPS 40 may communicate with a clinical trial database 60, whether internally or externally disposed in relation thereto. The clinical trial database 60 stores information defining a clinical trial and a plurality of anonymous patients, such as patient 70, participating in the clinical trial. For example, as depicted in the embodiment shown in FIG. 1A, the clinical trial database 60 may store key-coded data 73, which may comprise datasets which do not carry personal identifiers. Such key-coded data 73 may or may not carry information pertinent to any particular patient and may comprise a variety of study and/or trial-specific information, such as study timeline information, sample drop-off and pickup schedules, visit schedules, and other such information associated with the progression of the study and/or trial. In at least one embodiment, such key-coded data 73 may additionally comprise a screening identifier 61, which may identify associated portions of data—e.g., a specific patient or patients within a given study and/or trial—without identifying the patient themselves. Such a screening identifier 61 may comprise, for instance, some unique identifier associated with each patient. In at least one embodiment, such a screening identifier 61 may be comprised of the combination of two separate identifiers, namely: (a) a study identifier 62, which may comprise some alphanumeric string used to identify a particular study and/or trial at issue, and which may generated by the sponsor of the study and/or trial; and (b) a reference identifier 63, which may comprise some unique alphanumeric string which is automatically and/or randomly generated by the clinical trial database 60. Accordingly, while the study identifier 62 is not guaranteed to be unique on its own, the combination thereof with the reference identifier 63 may form a unique screening identifier 61 which may be used to identify particular patients of particular studies and/or trials. Accordingly, it may be understood the clinical trial database 60 may not store or otherwise access any identifying patient data 71, as will be discussed in greater detail hereafter.

With continued reference to FIGS. 1A and 1B, it may be seen the PPS 40 may also communicate with the intermediary system 30 for processing supply orders (e.g., supply or sample kits to be shipped to the patient 70, sample pickups from the patient 70, etc.). Such an intermediary system 30 may comprise an intermediary database 31 in at least one embodiment of the present invention. In such an embodiment, the intermediary database 31 may comprise an internally firewalled system that is operated by the same entity as the PPS 40, but which is distinct therefrom. As such, the intermediary database 31 may be configured to control access to the data stored therein. Accordingly, such an intermediary system 30 may operate to prevent the PPS 40 from accessing certain portions of data through, for instance, the private key 85 stored therein, as previously discussed heretofore.

More specifically, such an intermediary system 30, and more pertinently the intermediary database 31 thereof, may be configured to store the obfuscated patient data 72 provided by the patient from the patient device 20. For instance, obfuscated patient data 72 may pass from the patient device 20, through the PPS 40, and into the intermediary database 31. And, in at least one embodiment, such an intermediary database 31 may additionally be configured to store a private key 85, which may be used to decrypt the obfuscated patient data 72 and obtain the identifying patient data 71. Such a private key 85 may be a counterpart to the registration code 83, and particularly the shared key 84 thereof, such that the private key 85 and the shared key 84 form a key pair. In so doing, it may be understood the intermediary system may function as a controller for the patient's data, such that the intermediary system 30 dictates who has access to the identifying patient data 71 stored within the intermediary database 31, thereby maintaining the blinding and other anonymity-based regulations applicable to the patient's data. In at least one embodiment, such as the one depicted in FIG. 1B, such registration code 83, shared key 84, and private key 85 may be generated according to the encoding algorithm 80 discussed heretofore.

However, in at least one alternative embodiment envisioned herein, such an intermediary system 30 and the intermediary database 31 thereof may instead comprise a third-party system that enables separation of patient data by storing, separate from the clinical trial database 60, the identifying patient data 71 of the patient 70, such as patient demographic information and other patient identifying information. For example, the intermediary system 30 may maintain an account associated with the patient 70 and store, in an intermediary database 31, an account record including at least one selected from the group consisting of data relating to the patient 70, such as the patient ID, the study ID, their name, mailing address, email address, phone number, date of birth, gender, or other demographic information akin thereto. The storage of the foregoing data may, in at least one embodiment, be made pursuant to the foregoing disclosure relating to the use of a pair of a shared key 84 and a private key 85 which operate to transform the obfuscated patient data 72 passed through the PPS 40 into the identifying patient data 71. In such embodiments, the PPS 40 may be blocked from accessing the account associated with the patient operated by the intermediary system 30. However, in alternative embodiments, the intermediary system 30 and/or intermediary database 31 may instead communicate directly with the PPS 40 in such a manner that the PPS 40 may access the account associated with the patient operated by the intermediary system 30, provided the intermediary system 30 controls access to the identifying patient data 71 of the patient 70 in such a manner that the PPS 40 does not have visibility to the same.

Accordingly, because the intermediary system 30 and/or intermediary database 31 may, in alternate embodiments, be owned and operated by the same entity as the one providing the PPS 40, or some other third-party, it may be understood various information security mechanisms may be implemented to ensure the separation of identifying patient data 70 from the PPS 40 and the clinical trial database 60. For example, a firewall may be used to control access to patient information. The stored patient information may also be encrypted, wherein only authorized systems and software applications can access the stored patient information in decrypted form, as discussed heretofore. Auditing functionality is also implemented to track accesses to the stored patient information, which may be needed to comply with regulatory requirements associated with the clinical trial.

With further reference to FIG. 1A, supply orders (e.g., a kit and/or drug delivery) requested by the patient 70 through the PPS 40 are provided to the intermediary system 30 before being subsequently passed along to the supply system 50. In so doing, the intermediary system 30, and particularly the intermediary database 31, may receive an order request along with the patient's 70 obfuscated patient data 72 and/or key-coded data 73. Once received, the intermediary database 31 may determine the pertinent identifying patient data 71 to be passed along to the supply system 50, such as the patient's 70 name and address. And, once such identifying patient data 71 is transmitted to the supply system 50, such supply system 50 may fulfill the order and ship the supply order to the patient 70.

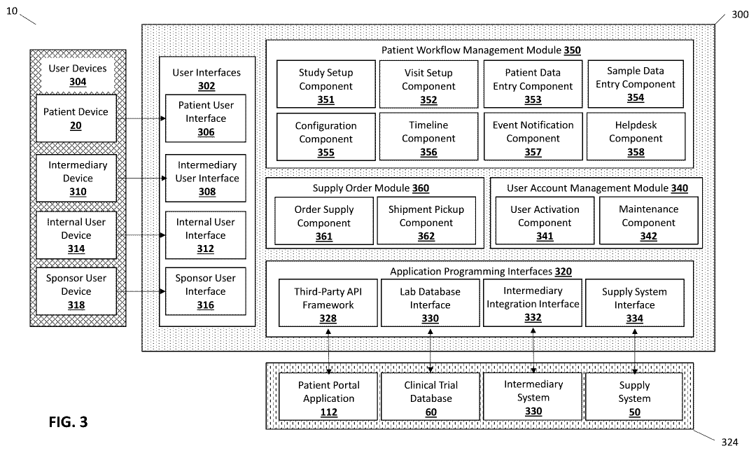

Alternatively, when the patient 70 requests a supply pickup (e.g., a kit pickup) through the PPS 40, the PPS 40 provides the pickup request to the intermediary system 30. The intermediary system 30 then provides pickup information and pertinent identifying patient data 71 to the supply system 50 or, in some instances, a courier service affiliated with the supply system 50 or intermediary system 30. The supply system 50 or courier service then picks up the kit from the home of the patient 70. The PPS 40 communicates with the patient 70, the intermediary system 30, and the supply system 50 through respective APIs 320, as depicted in FIG. 3 and discussed in greater detail hereafter. However, a variety of alternative solutions for enabling communication between the PPS 40, the patient 70, the intermediary system 30, and/or the supply system 50 may be used in various embodiments of the present invention, as may be understood by those having ordinary skill in the art.

As previously noted, the PPS 40 may be implemented on one or more computing devices, such as, for example, one or more servers, and includes a plurality of hardware and software components. FIG. 3 illustrates software components 300 included in the PPS 40, according to some examples. For instance, it may be seen the PPS 40 is configured to provide a plurality of user interfaces 302 to a plurality of user devices 304. For example, the PPS 40 provides a patient user interface 306 to the patient device 20, an intermediary user interface 308 to one or more intermediary devices 310 included in the intermediary system 30 and which may provide a site-level view of all data—e.g., identifying patient data 71, obfuscated patient data 72, registration code 83 and/or key-coded data 73—, an internal user interface 312 to one or more internal user devices 314 of the PPS 40, and a sponsor user interface 316 to one or more clinical trial sponsor devices 318. The PPS 40 may provide the patient user interface 306 to the patient device 20 through the patient portal application 112 installed on the patient device 20. Such a patient portal application 112 may be configured for use in connection with a variety of operating systems such as, without limitation, Microsoft's Windows, Google Android, or Apple IOS. The PPS 40 may respectively provide the intermediary user interface 308, the internal user interface 312, and the sponsor user interface 316 through a web browser installed on respective ones of the one or more intermediary devices 310, the one or more internal user devices 314, and the one or more clinical trial sponsor devices 318, or through other similar means as understood by those having ordinary skill in the art.

The PPS 40 may further include a plurality of APIs 320 for exchanging data with a plurality of external systems 324. The plurality of APIs 320 may include, for example, a third-party application integration framework 328, a lab database interface 330, an intermediary integration interface 332, and a supply system interface 334. The third-party application integration framework 328 enables the patient device 20 to launch a third-party application (e.g., an application associated with the intermediary system 30) from the patient portal application 112 for communication therewith. For example, the lab database interface 330 enables the PPS 40 to exchange information with clinical trial database 60 for exchanging information relating to the clinical trial (e.g., sample processing status, missing sample status, etc.). The PPS 40 communicates with the intermediary system 30 using the intermediary integration interface 332 and communicates with the supply system 50 through the supply system interface 334. The intermediary system 30 may communicate with the supply system 50 through means separate from the PPS 40 (e.g., another API). In so doing, it may be the understood the patient(s) 70 may provide identifying patient data 71 directly to the third-party applications without contaminating the patient portal system 40. However, as previously discussed, alternative methods for facilitating communication between the plurality of external systems 324 is envisioned herein, as understood by those having skill in the art.

The PPS 40 may further include a user account management module 340 for controlling registration of the patient 70 with the PPS 40 and management of the patient's profile within the system (at least portions of such profile the PPS 40 is authorized to access and use). Such a user account management module 340 may comprise a user activation component 341, which may enable a patient 70 to register their patient device 20. As may be understood, in at least one embodiment, such a user activation component 341 may thus be configured to facilitate use of the encoding algorithm 80 by the patient device 20. Meanwhile, a maintenance component 342 may be configured to enable users to provide roles and other maintenance-related operations relevant to users of the PPS 40.

The PPS 40 may also include a patient workflow management module 350 and a supply order module 360. The patient workflow management module 350 provides general functionality of the patient portal application 112. For example, the patient workflow management module 350 may provide study timeline information, notifications, reminders, past visit information, and patient profile information to the patient 70 through the patient portal application 112. The patient workflow management module 350 may also receive from the patient 70, through the patient portal application 112, input relating to scheduling a visit (e.g., a kit pickup visit, a kit delivery visit, or an in-person visit). The patient workflow management module 350 may also connect the patient 70 with an investigator or a helpdesk.

Likewise, the patient workflow management module 350 may enable the real-time collection of clinical sample data via the provision of one or more sample collection forms which may comprise various prompts and/or questions indicative of the clinical sample data to be collected. Such sample collection forms may be integrated directly with the PPS 40 and/or the clinical trial database 60 such that the clinical sample data may be readily reviewed and verified in real-time. Accordingly, in at least one embodiment, such sample collection forms may be provided to the patient 70 through the patient portal application 112. In at least one embodiment, the patient workflow management module 350 may be interconnected with some system and/or database, whether comprising the clinical trial database 60 or otherwise, which is configured to generate such sample collection forms according to the specifics of the protocol of the clinical trial at issue; however, it may be understood the generation of such sample collection forms by the PPS 40 is contemplated herein.

In view thereof, it may be understood the patient workflow management module 350 may comprise a variety of components configured to enable those users involved with performing the clinical trial to manage all data, timelines, and other tasks associated therewith. For instance, such a patient workflow management module may comprise: (a) a study setup component 351 configured to enable users to configure appropriate settings to align with a given clinical trial; (b) a visit setup component 352 configured to enable users to configure visits for patients at clinical trial sites; (c) a patient data entry component 353 configured to enable users to enter various patient data relevant to the clinical trial; (d) a sample data entry component 354 configured to enable users to enter sample collection data; (e) a configuration component 355 configured to enable users to configure and customize their settings; (f) a timeline management component 356 configured to enable users to access, review, and change information relating to a timeline of the trial; (g) an event notification component 357 configured to enable a user to review and apply notifications for various clinical trial events; and (h) a helpdesk component 358 configured to enable a user to utilize and/or connect a patient 70 with a helpdesk or other similar operator who may assist in operation of the DCT system 10 and/or PPS 40.

With continued reference to FIG. 3, the supply order module 360 of the PPS 40 is configured to receive and manage supply delivery orders and supply pickup orders from the patient 70. For example, the supply order module 360 receives requests from the patient 70 for a supply kit to be delivered and provides delivery request information to the intermediary system 30 and the supply system 50 for fulfillment of the request, such as through an order supply component 361. The supply order module 360 may also provide the delivery request information to an investigator device—e.g., internal user device 314—for review and approval of the request. Similarly, the supply order module 360 receives requests from the patient 70 for a supply kit to be picked up (e.g., after a sample has been collected by the patient 70) and provides pickup request information to the intermediary system 30 and the supply system 50 for fulfillment of the request, such as through a shipment pickup component 362. The PPS 40 further includes a patient portal application database 370 for storing patient portal application information (e.g., for interfacing with the patient device 20 via the patient portal application 112). In at least one embodiment of the present invention, such supply system 50 may be configured to include one or more vendors configured to fulfill the request in addition to a variety of couriers and other shipping and/or postal servicers around the world.

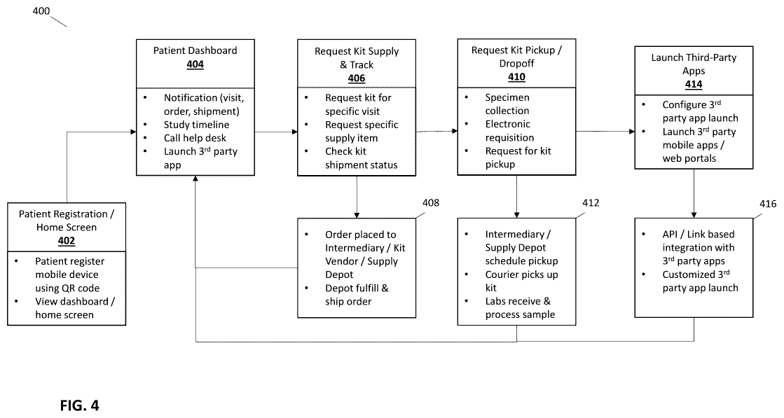

FIG. 4 is a flowchart of a patient portal application workflow method 400 according to at least one embodiment. In one example, the method 400 is implemented using components of the PPS 40, such as, for example the user account management module 340, the patient workflow management module 350, and the supply order module 360. The method 400 is described below with continued reference to FIGS. 1-3.

The method 400 includes the PPS 40 receiving a registration request from the patient 70 to register the patient device 20 (in block 402). In one embodiment, the patient 70 registers the patient device 20 through a registration code 83. In at least one embodiment, such a registration code may comprise a quick response (“QR”) code, or alternatively a bar code, which may be scanned by the camera 110 of the patient device 20. Such registration code 83 may be associated with a specific clinical trial in which the patient 70 is enrolled and, as discussed heretofore, may comprise a shared key 84 configured to enable the application of the encoding algorithm 80 to the data provided by the patient 70. For example, when a patient (or the patient's physician or trial investigator) desires to enroll in a clinical trial, the investigator may request a spot in the clinical trial via one or more systems or software applications (e.g., by entering or generating a screening identifier, a study identifier, and a reference identifier to register the patient into PPS 40 and/or some other interconnected lab data portal representing an external portal for clients and sites to view and manage study data in real-time). Upon receipt of such request by the investigator, the PPS 40 may generate the registration code 83 through, for instance, the account management module 340 and/or the encoding algorithm 80. In response to receiving approval, the investigator provides the registration code 83 to the patient 70, whether in a physical, tangible form or through some electronic means such as text or email, wherein such registration code 83 is unique to the patient 70 and the particular clinical trial in which the patient 70 intends to enroll. Hence, the registration code 83 may be provided to the patient 70 without requiring a physical visit to a site, whether an investigator site at which the patient's 70 treatment is managed, or otherwise. The registration code 83 may be provided with additional instructions, including instructions for downloading the patient portal application 112 as described below.

The patient 70 may use the registration code 83 to register his or her user device with the PPS 40 and access user interfaces described herein for ordering kits, scheduling sample collection and pick-ups, and the like. For instance, as previously discussed, the registration code 83 and the shared key 84 thereof, may enable the patient 70 to provide their obfuscated patient data 72 to the PPS 40, which may then transmit any relevant information to the intermediary system 30 and/or intermediary database 31 for further actions relating thereto. Upon the patient's 70 registration of their patient device 20 via the registration code 83, the registration code 83, and the shared key 84 thereof may be stored within the intermediary database 31 along with the associated private key 85. In so doing, the intermediary database 31 may therefore access the identifying patient data 71 from the obfuscated patient data 72 transmitted thereto by the PPS 40. As such, the intermediary system 30 and/or intermediary database 31 may enable kit shipments and deliveries for a particular clinical trial using the patient's 70 identifying patient data 71, such as their name and address, without providing such identifying patient data 71 to the PPS 40 and/or clinical trial database 60. As may be understood, the patient 70 may not directly communicate with the intermediary system 30 and/or intermediary database 31 in certain embodiments; however, in alternative embodiments, it is contemplated the patient 70 may communicate directly with the intermediary system 30 and/or intermediary database 31 via certain authentication schemes, such as single sign-on procedures. In instances wherein a patient 70 is involved in two or more clinical trials, whether at the same or at different times, such a patient 70 will receive two or more registration codes 83 to be applied to their data. In so doing, the identifying patient data 71, obfuscated patient data 72, and key-coded data 73 associated with such patient 70 may be appropriately segmented for each trial in which the patient 70 participates.

In at least one alternative embodiment, a patient 70 may register the patient device 20 with the PPS 40 by downloading the patient portal application 112 to the device 20 (if not already downloaded) and, within the application, scanning the received registration code 83. Once the registration code 83 is received, the patient portal application 112 may identify at least one unique device identifier for the patient device 20 (e.g., a machine or hardware identifier or code, such as a media access control (MAC) address and/or internet protocol (IP) address) and record the same to the PPS 40. The PPS 40 may record such device identifier of the patient device 20 with the associated registration code 83. In so doing, it may be understood the unique device identifier for the patient device 20 may be used similarly to the shared key 84, such that the same is linked with a private key 85 stored within the intermediary database 31. In other words, rather than creating a standard user account or profile with the patient 70, which would require requesting identifying information, contact information, log-in information, and the like through the application 112, the PPS 40 may instead use the unique device identifier of the patient device 20 as the registration link between the device 20 and the PPS 40. Each time the patient portal application 112 installed on the device 20 communicates with the PPS 40, the portal application 112 provides the unique device identifier to the PPS 40 for the subsequent transmittal to the intermediary database 31. Thus, no identifying patient data 71 is ever received or maintained by the PPS 40 (or the patient portal application 112), which ensures regulatory requirements are complied with; rather, the PPS 40 only receives and transmits the obfuscated patient data 72. In some embodiments, rather than providing a unique hardware code or address to identify the patient device 20, the patient portal application 112 may instead generate a unique device identifier for the device 20 (e.g., based on a unique hardware or software code or address associated with the device 20), which further obscures the connection between the patient device 20 and a particular patient 70.

With continued reference to FIG. 4, after registering the patient device 20, the PPS 40 provides a patient dashboard user interface to the patient device 20 through the patient portal application 112 (in block 404). The patient dashboard user interface may provide study timeline information, such as a list and summary of previous and future visits. The patient dashboard user interface may also provide alerts or notifications relating to outstanding action items (e.g., missing samples), an upcoming sample collection deadline, or an upcoming delivery. The patient dashboard user interface may additionally connect the patient 70 to a helpdesk or an investigator, such as through the patient workflow management module 350 of the PPS 40. The information provided through the patient portal application 112 is unique to the patient 70 (e.g., provides information only regarding the clinical trial(s) in which the patient is enrolled).

The method 400 also includes the PPS 40 receiving order information associated with a requested kit order from the patient 70 through the patient portal application 112 (in block 406). In response to receiving the order information, the PPS 40 communicates with the intermediary system 30 and the supply system 50 to fulfill the requested order (in block 408). For example, the PPS 40 may provide the order information (e.g., an identifier of a particular type of kit) and a patient ID corresponding to the patient 70 (e.g., the obfuscated patient data 72 and/or key-coded data 73) to the intermediary system 30. The intermediary system 30 may then determine and provide the identifying patient data 71, such as the patient's 70 name, mailing address, and the order information to the supply system 50 for arranging delivery of the requested kit. The identifying patient data 71, such as the patient's 70 mailing address, however, is never stored by the PPS 40, but instead passes therethrough as obfuscated patient data 72.

The method 400 also includes receiving a kit pickup request from the patient 70 through the patient portal application 112 (in block 410). In response to receiving the kit pickup request, the PPS 40 provides instructions through the application 112 on specimen collection and provides an electronic requisition (“e-requisition”) form. The PPS 40 communicates with the intermediary system 30 and the supply system 50 for scheduling the pickup of the kit by providing, for example, the patient ID and the e-requisition form to the intermediary system 30 and/or the supply system 50 (in block 412). In some instances, the intermediary system 30 provides kit pickup instructions to a courier service affiliated with the intermediary system 30. After the kit is picked up by the supply system 50 or the courier service, the kit is delivered to a lab so that the sample collected by the patient 70 may be processed.

The method 400 depicted in FIG. 4 may additionally include the PPS 40 receiving a request from the patient 70 to launch a third-party application from the patient portal application 112 (in block 414). As previously discussed, the third-party application may be another application other than the patient portal application 112 used in the decentralized clinical trial environment. For example, the third-party application may be an application associated with the intermediary system 30. In response to receiving the request to launch the third-party application, the PPS 40 launches the third-party application using, for example, the third-party application integration framework 328 of the PPS 40 (in block 416). Such third-party applications may comprise a variety of systems including applications to manage patient dosing and/or delivery, document repository applications, applications configured for scheduling appointments with doctors and/or clinical trial sites, and other similar applications generally relating to clinical trials.

FIG. 5 is a flowchart of a patient portal registration method 500 according to at least one embodiment. In depicted example, the method 500 is implemented using components of the PPS 40, such as the user account management module 340. The method 500 is described below with continued reference to FIGS. 1-3.

The method 500 includes receiving a new patient registration request from an intermediary device (e.g., from an investigator) for the patient 70 (in block 504). The new patient registration request includes, for example, a screening ID of the patient 70. In some instances, the new patient registration request also includes a year of birth of the patient, a date of birth of the patient, and/or a gender of the patient. The method 500 may also include receiving a request for re-registering the patient 70, for example, if the patient device 20 has changed (in block 506). In response to receiving the registration or re-registration request, the PPS 40 generates one or more registration codes 83, such as one or more QR codes, one or more bar codes, or another scannable code as described above (in block 508). The one or more registration codes 83 may be printed and provided to the patient 70 or be provided to the intermediary system 30 and electronically provided to the patient 70 (e.g., via text or email). In some instances, the PPS 40 generates a first registration code 83, or an application installation code, representing first key-coded data 73 for enabling the patient 70 to install the patient portal application 112 on the patient device 20 (in block 512), and generates a second registration code 83, or a trial code, representing second key-coded data 73 for enabling the patient 70 to register in the patient portal application 112 for a particular clinical trial (in block 514). The PPS 40 may register the patient 70 by generating a shared key 84 for the patient 70 within the clinical trial. As described above, the shared key 84 may be used to apply the encoding algorithm 80 to the data provided by the patient 70, such that the PPS 40 cannot store or access any identifying patient data 71. In some embodiments, the intermediary system 30 also receives the unique device identifier (in block 516) and uses the device identifier or a different unique identifier for the patient 70 and/or the patient device 20 as the shared key 84 with the PPS 40.

FIG. 6 is a flowchart of a supply order method 600 according to an embodiment. In one example, the method 600 is implemented using components of the PPS 40, for example, the supply order module 360. The method 600 is described below with continued reference to FIGS. 1-3.

The method 600 includes receiving, from the patient 70 via the patient portal application 112, a request to access study or visit information (in block 604). The PPS 40 may provide, for example, a user interface to the patient device 20 including a list of actions items such as scheduling a visit for dropping off a sample, placing an order, or scheduling a sample pickup. As noted above, the provided information may be unique to both the patient 70 and the clinical trial in which the patient 70 is enrolled. For example, the PPS 40 may provide sample collection information including an identifier of a particular supply kit available for order by the patient 70. In at least one embodiment, the information relating to the supply kit required by the patient may be automatically populated and added to an order within the patient portal application 112, such as through the order supply management component of the supply order module 360. In such an embodiment, verification and/or approval of the supply kit automatically populated in the patient portal application 112 may be required by the patient's physician and/or trial investigator before the patient may order the same. Accordingly, although the PPS 40 could provide a generic user interface for ordering various types of kits, errors could be introduced if a patient did not order the correct kit for their particular clinical trial or ordered kits or provided samples in a way that did not comply with the trial timeline. Accordingly, by using the key-coded data 73 as described herein, the PPS 40 can identify what particular kits, pick-ups, etc. to offer the patient 70 within the application 112 and communicate the same to the intermediary system 30 without gaining access to the identifying patient data 71.

The method 600 also includes receiving a selection of a supply kit and/or a specific supply item from the patient 70 via the patient portal application 112 (in block 608). For example, the patient 70 may select a single item that may be included in a supply kit. The PPS 40 may provide a notification to a device included in the intermediary system 30 for approval of the selection, for example, by providing the notification via the intermediary user interface 308. In response to receiving, from the intermediary system 30, approval for the selection (in block 612), the PPS 40 generates an order request for the selected kit and/or specific item, and transmits, through the intermediary integration interface 332, an instruction to the intermediary system 30 to initiate shipment of the selected supply kit. (in block 616). After sending the order request to the intermediary system 30, the PPS 40 may receive a response from the intermediary system 30 confirming that order request has been received, and that the intermediary system 30 has placed the order with the supply system 50 (in block 620). The order placed with the supply system 50 includes the patient details needed for placing the supply order, for example, patient name and address. The PPS 40 may receive a confirmation from the intermediary system 30 confirming that the supply system 50 has received the order (in block 624). The PPS 40 may also receive a shipment confirmation from the intermediary system 30 after shipment of the order by the supply system 50 to the patient 70 (in block 628). The PPS 40 may also provide, to the patient device 20, order status information during the order process (in block 634). For example, using the patient portal application 112, the patient 70 may view an order processing status and/or a shipment status. The shipment status may include the expected delivery date or other tracking information, such as that provided by some third-party courier service. For example, intermediary system 30 may receive tracking information from the supply system 50, and provide the tracking information to the patient 70, for example, through the third-party application.

FIG. 7 is a flowchart of a sample kit pickup method 700 according to an embodiment. In one example, the method 700 is implemented using components of the PPS 40, for example, the supply order module 360. The method 700 is described below with continued reference to FIGS. 1-3.

The method 700 includes receiving, from the patient 70 via the patient portal application 112, a request to access study or visit information (in block 704). The PPS 40 may provide, for example, a user interface to the patient device 20 including a list of actions items such as placing an order, scheduling a visit for dropping off a kit, or scheduling a kit pickup. In response to receiving, from the patient device 20, a selection to drop off a kit or have a kit picked up (in block 708), the PPS 40 provides an e-requisition form to the patient 70 through the patient portal application 112 (in block 712). The patient 70 may provide, on the e-requisition form, a sample kit ID associated with the sample kit, a scheduled pickup date, or a scheduled pickup time. The PPS 40 may further provide sample collection instructions to the patient 70 through the patient portal application 112. If, at block 708, the patient 70 selected to drop off the sample kit, the patient 70 drops off the sample kit at a drop-off location, such as for example, a courier service or the supply system 50 (in block 714). If at block 708, the patient 70 selected a sample kit pickup, the PPS 40 generates a sample kit pickup request, and sends the sample kit pickup request to the intermediary system 30 (in block 716). The sample kit pickup request includes, for example, the patient ID, a requisition ID, a pickup ID, the e-requisition form, and/or a subset of information included in the e-requisition form. The intermediary system 30 may provide a confirmation of receipt of the request to the PPS 40, and places a pickup order with the supply system 50 or a courier service with the patient name and address (in block 720). The PPS 40 may receive a confirmation from the courier service or the supply system 50 via the supply system interface 334, and/or the intermediary system 30 when the kit pickup order is received by the courier service or supply system 50 (in block 724). The PPS 40 may receive a confirmation from the courier service or the supply system 50 via the supply system interface 334, and/or the intermediary system 30 when the courier service or supply system 50 has pickup up the kit from the patient's home (in block 724). The PPS 40 may provide, to the patient device 20, kit pickup status information during the kit pickup process (in block 734). For example, the PPS 40 may provide one or more notifications to the patient device 20 including an expected pickup date of the sample kit.

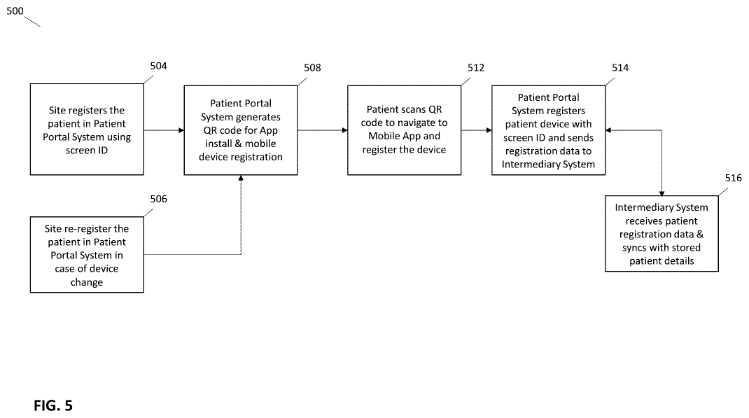

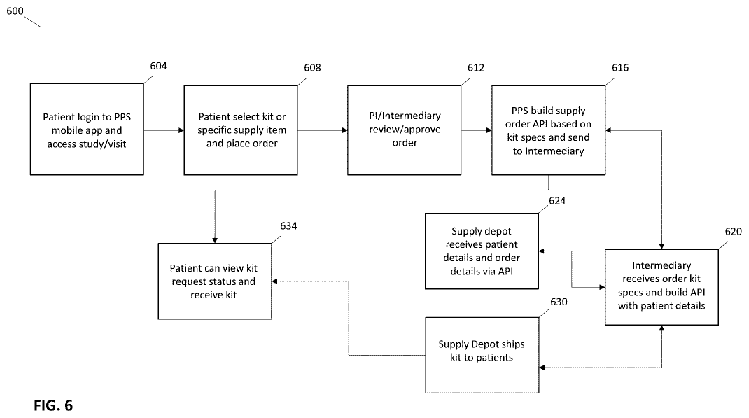

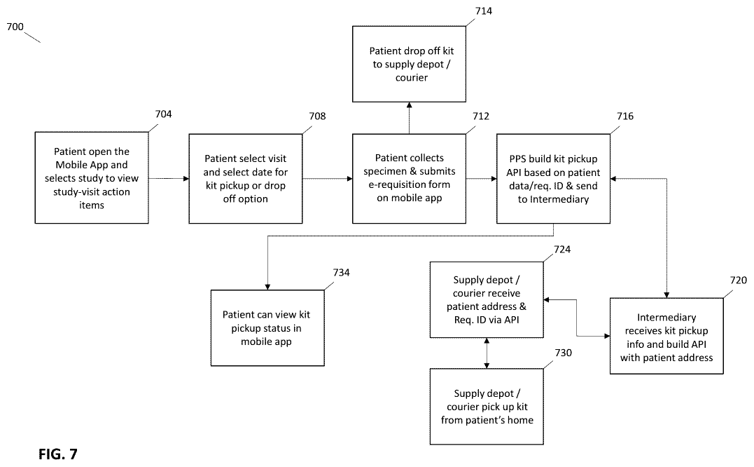

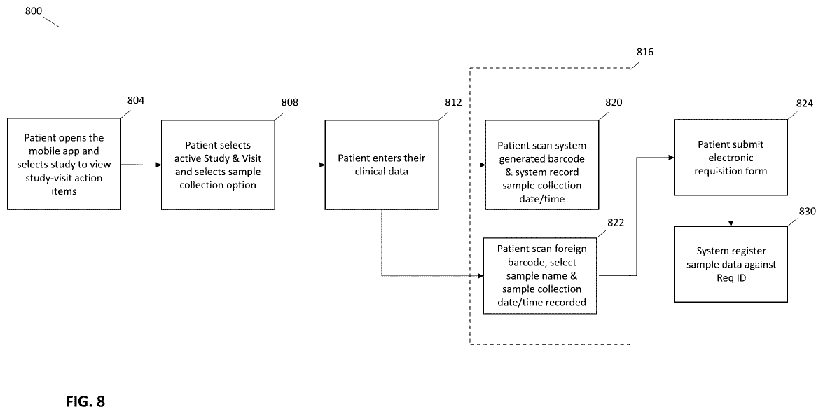

FIG. 8 is a flowchart of an electronic requisition method 800 according to an embodiment. In one example, the method 800 is implemented using components of the PPS 40, for example, the supply order module 360. The method 800 is described below with continued reference to FIGS. 1-3.

The method 800 includes receiving from the patient 70 via the patient portal application 112, a request to access study or visit information (in block 804). The PPS 40 may provide, for example, a user interface to the patient device 20 including a list of actions items such as placing an order, scheduling a visit for dropping off a kit, or scheduling a kit pickup. In response to receiving, from the patient device 20, a selection to drop off a kit or have a kit picked up (in block 708), the PPS 40 prompts the patient 70 to enter clinical trial information associated with the patient 70 (e.g., a clinical trial ID, a patient ID, a kit ID associated with the sample kit to be picked up, or the like) (in block 808), and provides a requisition form user interface to the patient device 20. Using the camera 110 and the patient portal application 112, the patient 70 scans a barcode included in the sample kit to be picked up, and the PPS 40 receives scanned barcode data based on the scanned barcode (in block 812). Based on the scanned barcode data, the PPS 40 may determine whether the barcode is generated by the PPS 40 (e.g., included in the clinical trial database 60). If the barcode is a PPS-generated barcode, the PPS 40 determines a sample name based on the scanned barcode data, and records, as requisition information, the date and time of the sample collection and the sample name (in block 820). If the barcode is not a PPS-generated barcode, the PPS 40 prompts the patient 70 to select a sample name (e.g., from a drop-down menu, using a text entry field, or the like), and records, as e-requisition information, the date and time of the sample collection and the sample name (in block 822). The e-requisition information may also include the scanned barcode data. The PPS 40 may prompt the patient 70 to review and submit the e-requisition information. After the PPS 40 receives a submission of the e-requisition information from the patient 70 via the patient portal application 112 (in block 824), the PPS 40 generates a requisition ID and registers, in the clinical trial database 60, sample accessioning data based on the e-requisition information and the requisition ID (in block 830).

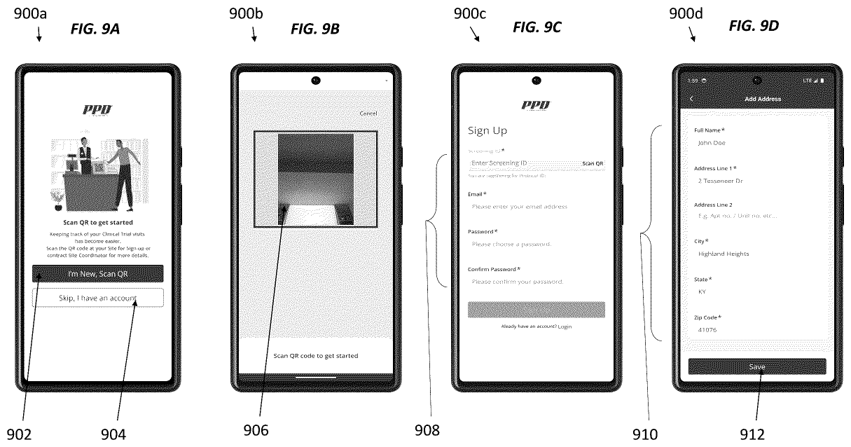

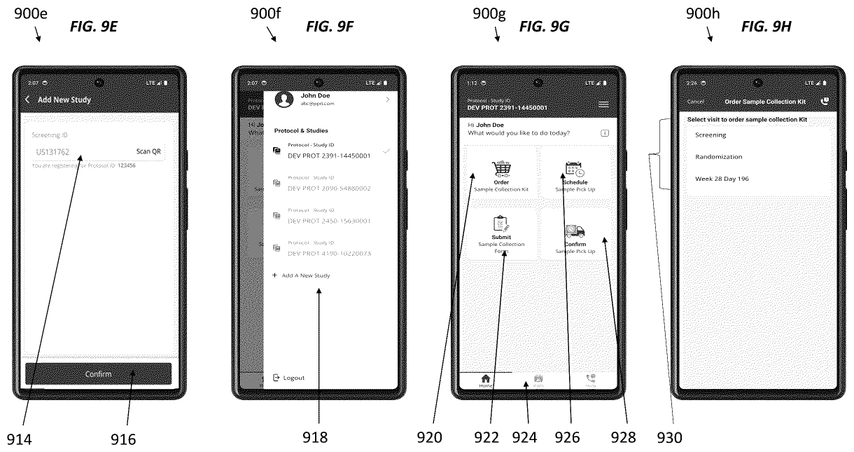

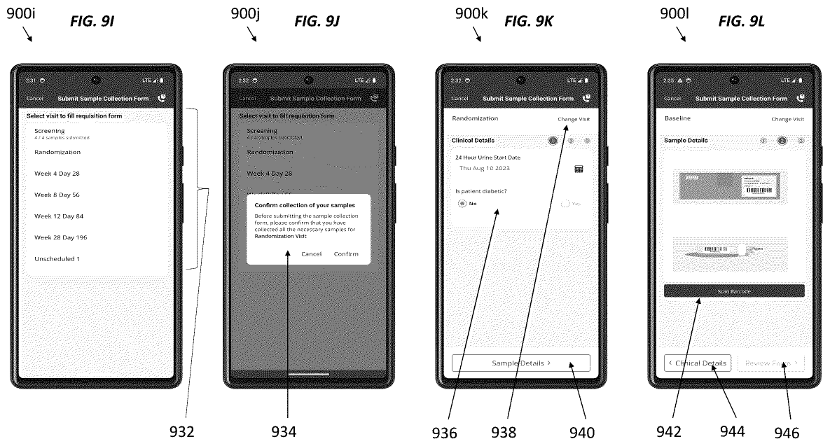

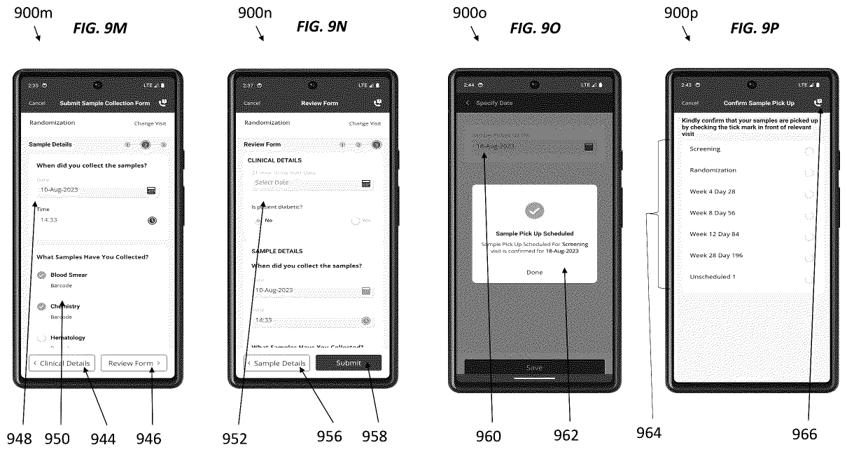

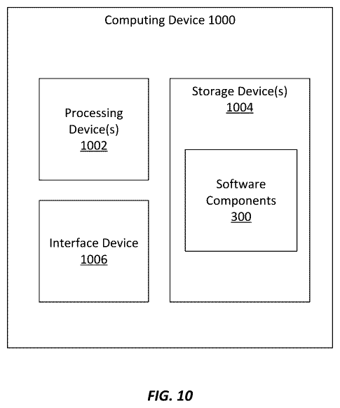

FIGS. 9A-9P depict a plurality of screens 900—i.e., graphical user interfaces—provided by the patient user interface 306 of the patient portal application 112 on the patient device 20. Accordingly, the various screens 900 of the patient user interface 306 serve to display and receive pertinent information to and from the patient 70.

For instance, FIGS. 9A-9D depict various screens 900 displayed during the registration process, such as during the method 500 depicted in FIG. 5. For instance, in FIG. 9A, the screen 900a includes a prompt informing the patient 70 of the applicable steps to register the patient device 20 with the PPS 40. Such a prompt may instruct the patient 70 to select a scan code button 902 or a skip button 904 depending on whether the patient 70 already has an account for the clinical trial at issue. Pushing the scan code button 902 may result in the display of the screen 900b shown in FIG. 9B, wherein an image capture zone 906 may be displayed to assist the patient 70 in scanning the registration code 83. Once the registration code 83 is scanned by the patient device 20 and transmitted to the PPS 40, the screen 900c depicted in FIG. 9C may be shown, wherein a plurality of registration fields 908 may be shown to the patient 70. Such plurality of registration fields 908 may comprise data fields into which the patient 70 may input data such as, without limitation, the applicable screening ID, the patient's 70 email address, and the patient's 70 password for the account. In at least one embodiment, the registration field 908 directed to the applicable screening ID may be automatically entered by the patient portal application 112 upon scanning the registration code 83. Once the patient 70 completes the plurality of registration fields 908, the patient 70 may be directed to the screen 900d depicted in FIG. 9D, through which the patient 70 may enter certain identifying patient data 71, namely, their address, through the plurality of address fields 912 available thereon. Once entered, the patient 70 may then select the save button 912 to complete the initial device registration process.

Likewise, FIGS. 9E-9F depict various screens 900 displaying by the patient portal application 112 which enable the patient 70 to add and/or select a study and/or trial to and/or from their account. For instance, the screen 900e depicted in FIG. 9E may comprise a screening ID field 914 through which a user may enter the applicable screening identifier 61 associated with the particular study and/or trial. In at least one embodiment, such screening ID field 914 may be automatically entered by the patient portal application 112 when the registration code 83 is initially scanned by the patient device 20. Once the screening identifier 61 is entered by the patient 70, the patient may use a save button 916 to add the associated study and/or trial to their account. In contrast, the screen 900f depicted in FIG. 9F may be displayed by the patient portal application 112 to enable the patient 70 to select a particular study and/or trial for which they would like to perform various functions. Accordingly, such a screen 900f may comprise a study sidebar 918 having one or more selectable fields, each of which will direct the patient 70 to a different study and/or trial in which they are presently participating.

Similarly, FIGS. 9G-9P depict various screens 900 shown by the patient portal application 112 which enable a patient 70 to perform various functions relating to any one study and/or trial, such as during the method 700 of FIG. 7. For instance, FIG. 9G shows a screen 900g which may comprise a home screen for a particular study and/or trial, through which a patient 70 may be directed to the appropriate screens 900 and/or forms required to complete various functions. As may be seen, such a screen 900g may comprise: (a) an order button 920, through which a patient 70 may order a sample kit; (b) a submit button 922, through which a patient 70 may submit a sample collection form; (c) a schedule button 926, through which a patient 70 may schedule a date and/or time for a sample to be collected; and (d) a confirm button 928, though which a patient 70 may provide confirmation of a sample collection. Such a screen 900g may further include a taskbar 924, which may itself comprise buttons enabling the patient 70 to schedule a visit to a particular site, or otherwise request help, whether relating to study and/or trial or the patient portal application 112 itself. Upon selecting the order button 920 on the screen 900g depicted in FIG. G, the patient 70 may be shown screen 900h, through which the patient 70 may perform actions relating to the ordering of a sample kit. For instance, screen 900h may comprise one or more visit order selection fields 930 enabling the patient 70 to select the type of visit for which a sample kit is required.

Selecting the submit 922 button on the screen 900g displayed in FIG. 9G, on the other hand, may result in the display of the screens 900 shown in FIGS. 9I-9N, which collectively depict the sample collection forms proffered by the patient workflow management module 350 of the PPS 40, and therefore comprise various prompts and/or questions indicative of the clinical sample data to be collected. For instance, the screen 900i of FIG. 9I may first display one or more visit collection selection fields 932 through which the patient 70 may input the type of visit at issue for the sample collection. Once the appropriate type of visit is selected from the visit collection selection fields 932, the screen 900j of FIG. 9J may be displayed to the patient 70. As may be seen, such screen 900j may comprise a confirmation window 934, through which the patient 70 will be prompted to provide their confirmation that they selected the correct visit from the visit collection selection fields 932.

Once confirmed, the screen 900k of FIG. 9K may be displayed to the patient 70. As may be understood, screen 900k may display different information, and include alternative questions and/or prompts depending on the visit selected and/or the study/trial at issue. In the embodiment depicted in FIG. 9K, it may be seen the screen 900k comprises a clinical detail field 936, through which the patient 70 may input the requested information through a variety of means, whether comprising selectable buttons, fillable data fields, or otherwise. Such a screen 900k may further include a change visit button 938 enabling a user to return to the screen 900i depicted in FIG. 9I to select a different visit from the visit collection selection fields 932. Once the appropriate information is entered into the clinical detail field 936, the patient 70 may use the sample details button 940 to proceed to the screens 900l, 900m shown in FIGS. 9L-9M. Such screen(s) 900l, 900m may comprise a sample detail screen, through which the patient 70 may input information relevant to the sample itself. In at least one embodiment, such screen 900l may display a sample scan button 942, through which the patient 70 may scan the applicable barcode from a sample collection apparatus. Likewise, the screen 900m of FIG. 9M may be shown to the patient 70, either in place of or in addition to the screen 900l of FIG. 9L, for the input of additional information relating to the sample to be collected. For instance, such a screen 900m may include a sample date field 948, into which the patient may input the date and/or time at which the sample was collected. In at least one embodiment, the sample date field 948 may be automatically entered when the sample scan button 942 is used to scan the applicable barcode from the sample collection apparatus. Likewise, such a screen 900m may additionally include a sample identification field 950, through which the patient 70 may identify the type of samples collected, whether through selectable options, fillable data fields, or otherwise. As may be seen, the patient 70 may use a clinical details button 944 and a review form button 946 to either return to the prior form or proceed to the ensuing form, respectively.

Upon pressing the review form button 946, the screen 900n shown in FIG. 9N may be displayed to the patient 70, through which the patient 70 may review all information submitted through the prior screens 900i-900m. Accordingly, such a screen 900n may comprise a completed form field 952 displaying all the information and/or data selected or otherwise submitted by the patient 70 in connection with the present sample collection request. In at least one embodiment, the completed form field 952 may be locked, such that the patient 70 cannot directly make any changes to the information displayed thereby. Upon review of the completed form field 952, the patient 70 may use the sample details button 956 to return to prior screens to correct any information, or otherwise use the submit button 958 to complete the sample collection request.

FIG. 9O and FIG. 9P respectively depict a screen 900o for scheduling the date and time of sample collection, as accessible from the schedule button 926 shown in the screen 900g depicted in FIG. 9G, and a screen 900p for providing confirmation of sample collection, as accessible from the confirm button 928 shown in the screen 900g depicted in FIG. 9G. As may be seen in FIG. 9O, such a screen 900o may comprise a sample pickup field 960, through which the patient 70 may select a date they would like a given sample to be retrieved. Such a sample pickup field 960 may, in at least one embodiment, comprise a date picker or some other widget enabling the selection of a date from a calendar view. As may be noted, other fields in the various screens 900o discussed heretofore, such as the sample date field 948 shown in the screen 900m depicted in FIG. 9M may likewise use such a date picker or some similar widget to perform the same such date selection function. Once the date is selected, the screen 900o may be configured to display a date confirmation window 962, which may provide confirmation information to the patient 70 with respect to the selected date for sample collection. The screen 900p depicted in FIG. 9P may be utilized after the collection of the sample to confirm the collection thereof. Accordingly, such a screen 900p may comprise one or more visit confirmation selection fields 964 enabling the patient 70 to easily confirm a sample required for a given visit was collected. Finally, it may be seen the various screens 900h-900p of FIGS. 9H-9P may each include a help button 966 through which the patient 70 may receive assistance relating to the study and/or trial or the patient portal application 112, as discussed heretofore.

Finally, as noted above, the PPS 40 may be implemented by one or more computing devices. FIG. 10 is a block diagram of a computing device 1000 that may perform some or all the scientific instrument support functions and/or methods disclosed herein, in accordance with various embodiments. In some embodiments, the PPS 40 may be implemented by a single computing device 1000 or by multiple computing devices 1000. Further, as discussed below, a computing device 1000 (or multiple computing devices 1000) that implement(s) the PPS 40 may be part of one or more of a user local computing device, a service local computing device, or a remote computing.

The computing device 1000 of FIG. 10 is illustrated as having a number of components, but any one or more of these components may be omitted or duplicated, as suitable for the application and setting. In some embodiments, some or all of the components included in the computing device 1000 may be attached to one or more motherboards and enclosed in a housing (e.g., including plastic, metal, and/or other materials). In some embodiments, some of these components may be fabricated onto a single system-on-a-chip (SoC) (e.g., an SoC may include one or more processing devices 1002 and one or more storage devices 1004). Additionally, in various embodiments, the computing device 1000 may not include one or more of the components illustrated in FIG. 10, but may include interface circuitry (not explicitly shown) for coupling to the one or more components using any suitable interface (e.g., a Universal Serial Bus (USB) interface, a High-Definition Multimedia Interface (HDMI) interface, a Controller Area Network (CAN) interface, a Serial Peripheral Interface (SPI) interface, an Ethernet interface, a wireless interface, or any other appropriate interface).

The computing device 1000 may include a processing device 1002 (e.g., one or more processing devices). As used herein, the term “processing device” may refer to any device or portion of a device that processes electronic data from registers and/or memory to transform that electronic data into other electronic data that may be stored in registers and/or memory. The processing device 1002 may include one or more digital signal processors (DSPs), application-specific integrated circuits (ASICs), central processing units (CPUs), graphics processing units (GPUs), crypto-processors (specialized processors that execute cryptographic algorithms within hardware), server processors, or any other suitable processing devices.

The computing device 1000 may include a storage device 1004 (e.g., one or more storage devices). The storage device 6004 may include one or more memory devices such as random-access memory (RAM) devices (e.g., static RAM (SRAM) devices, magnetic RAM (MRAM) devices, dynamic RAM (DRAM) devices, resistive RAM (RRAM) devices, or conductive-bridging RAM (CBRAM) devices), hard drive-based memory devices, solid-state memory devices, networked drives, cloud drives, or any combination of memory devices. In some embodiments, the storage device 1004 may include memory that shares a die with a processing device 1002. In such an embodiment, the memory may be used as cache memory and may include embedded dynamic random-access memory (eDRAM) or spin transfer torque magnetic random-access memory (STT-MRAM), for example. In some embodiments, the storage device 1004 may include non-transitory computer readable media having instructions thereon that, when executed by one or more processing devices (e.g., the processing device 1002), cause the computing device 1000 to perform any appropriate ones or portions of the methods disclosed herein. The storage device 1004 may store some or all of the software components 300 of the PPS 40 described above with reference to FIG. 3.

The computing device 1000 may include an interface device 1006 (e.g., one or more interface devices 6006). The interface device 1006 may include one or more communication chips, connectors, and/or other hardware and software to govern communications between the computing device 1000 and other computing devices. For example, the interface device 1006 may include circuitry for managing wireless communications for the transfer of data to and from the computing device 1000. The term “wireless” and its derivatives may be used to describe circuits, devices, systems, methods, techniques, communications channels, etc., that may communicate data through the use of modulated electromagnetic radiation through a nonsolid medium. The term does not imply that the associated devices do not contain any wires, although in some embodiments they might not. Circuitry included in the interface device 1006 for managing wireless communications may implement any of a number of wireless standards or protocols, including but not limited to Institute for Electrical and Electronic Engineers (IEEE) standards including Wi-Fi (IEEE 802.11 family), IEEE 802.16 standards (e.g., IEEE 802.16-2005 Amendment), Long-Term Evolution (LTE) project along with any amendments, updates, and/or revisions (e.g., advanced LTE project, ultra mobile broadband (UMB) project (also referred to as “3GPP2”), etc.). In some embodiments, circuitry included in the interface device 1006 for managing wireless communications may operate in accordance with a Global System for Mobile Communication (GSM), General Packet Radio Service (GPRS), Universal Mobile Telecommunications System (UMTS), High Speed Packet Access (HSPA), Evolved HSPA (E-HSPA), or LTE network. In some embodiments, circuitry included in the interface device 1006 for managing wireless communications may operate in accordance with Enhanced Data for GSM Evolution (EDGE), GSM EDGE Radio Access Network (GERAN), Universal Terrestrial Radio Access Network (UTRAN), or Evolved UTRAN (E-UTRAN). In some embodiments, circuitry included in the interface device 7006 for managing wireless communications may operate in accordance with Code Division Multiple Access (CDMA), Time Division Multiple Access (TDMA), Digital Enhanced Cordless Telecommunications (DECT), Evolution-Data Optimized (EV-DO), and derivatives thereof, as well as any other wireless protocols that are designated as 3G, 4G, 5G, and beyond. In some embodiments, the interface device 1006 may include one or more antennas (e.g., one or more antenna arrays) to receipt and/or transmission of wireless communications.

In view of the above, it may be understood the various embodiments of the present disclosure are configured to resolve a variety of problems in the art related to existing online ordering technology in a decentralized clinical trial setting. Specifically, the various systems and processes disclosed herein may collectively enable anonymity-based data control solutions between the various systems and databases of a clinical trial, while allowing a patient to receive medications and other trial-related kits to be delivered and/or picked-up from their residence, thereby facilitating increased trial complexity while reducing patient burden.

1. A method for protecting personal patient information in a decentralized clinical trial, the method comprising:
registering a patient portal application on at least one user device via a registration code, the registration code comprising a shared key configured to generate obfuscated patient data for transmittal to a patient portal system via at least one server, the patient portal system communicatively configured in connection with an intermediary system having an intermediary database and a clinical trial database;
receiving, at the user device from the server, a sample collection information, the sample collection information including an identifier of a supply kit available for order by at least one patient participating in the clinical trial;
providing, at the user device, the sample collection information within a user interface of the patient portal application;
receiving, at the patient portal system, the obfuscated patient data relating to the provided sample collection information from the user device;
transmitting the obfuscated patient data from the patient portal system to the intermediary database, the intermediary database comprising a private key configured to access identifying patient data from the obfuscated patient data; and
transmitting the identifying patient data from the intermediary database to at least one supply system, the at least one supply system configured to fulfill an order in relation to the sample collection information.

2. The method of claim 1, wherein the registration code comprises a quick response (QR) code.

3. The method of claim 1, wherein the shared key of the registration code employs an obfuscation protocol and an encryption protocol to generate the obfuscated patient data.

4. The method of claim 1, wherein the obfuscated patient data is configured in a non-human readable format.

5. The method of claim 1, wherein the obfuscated patient data is configured in a manner that is not readable by the patient portal system.

6. The method of claim 1, wherein the identifying patient data transmitted from the intermediary database to the at least one supply system comprises a name and an address for the at least one patient.

7. The method of claim 1, further comprising receiving, at the user device, at least one notification from the server, the at least one notification related to a sample collection deadline.

8. A method for protecting personal patient information in a decentralized clinical trial, the method comprising:
placing a patient portal system in communicative configuration with a clinical trial database, an intermediary database, and at least one user device through at least one server;
storing, at the clinical trial database, key-coded data relating to at least one patient;
registering the at least one user device with the patient portal system through a patient portal application disposed thereon, such registration effectuated by at least one registration code provided to the at least one patient, receiving, by the patient portal application, data relating to the at least one patient and generating obfuscated patient data therefrom according to an encoding algorithm;
transmitting the obfuscated patient data through the patient portal system and to the intermediary database, the intermediary database having at least one private key stored therein, the at least one private key configured to access identifying patient data from the obfuscated patient data; and
transmitting the obfuscated patient data from the intermediary database to at least one supply system communicatively configured in connection therewith.

9. The method of claim 8, wherein the at least one registration code comprises a shared key linked to the private key, the shared key configured to apply the encoding algorithm to generate the obfuscated patient data.

10. The method of claim 9, wherein the encoding algorithm comprises an obfuscation protocol configured to apply at least one obfuscation technique to the data relating to the at least one patient and an encryption protocol configured to apply at least one encryption technique to the data relating to the at least one patient.

11. The method of claim 8, wherein the intermediary database resides within an intermediary system, such intermediary system comprising an internally firewalled system distinct from the patient portal system and the clinical trial database.

12. The method of claim 8, wherein the intermediary database resides within an intermediary system, such intermediary system comprising a third-party system.

13. A system for controlling data access to a patient's personal information in a decentralized clinical trial, comprising:
a patient portal system communicatively configured in connection with at least one patient device, a clinical trial database, and at least one intermediary database;
said at least one patient device comprising a processor communicatively configured in connection with a memory to a patient in input/output communication with said patient portal system through a patient portal application disposed thereon;
said at least one patient device configured to receive at least one registration code, said at least one registration code configured to enable access by said at least one patient device to said patient portal application for the input of identifying patient data therein;
said registration code configured to apply an encoding algorithm via a shared key to said identifying patient data, said encoding algorithm configured to transform said identifying patient data into obfuscated patient data and key coded data;
said patient portal system configured to receive said obfuscated patient data and said key coded data from said at least one patient device through said patient portal application;
said patient portal system configured to provide said key-coded data to said clinical trial database;
said patient portal system configured to provide said obfuscated patient data to said intermediary database;
said intermediary database configured to apply a private key stored therein to said obfuscated patient data to access said identifying patient data therefrom; and
said intermediary database configured to pass said identifying patient data to a supply system communicatively configured in connection therewith.

14. The system of claim 13, wherein said registration code comprises a quick response code.

15. The system of claim 13, wherein said identifying patient data comprises the patient's name and address.

16. The system of claim 13, wherein said obfuscated patient data is configured in a format unreadable by said patient portal system.

17. The system of claim 13, wherein said intermediary database comprises an internally firewalled system operated by the same entity as said patient portal system.

18. The system of claim 13, wherein:
said patient portal system is further configured to receive order information from said at least one patient device and provide said order information to said intermediary database;
said intermediary database is further configured to provide said order information to said supply system; and
said supply system is configured to fulfill said order information according to said identifying patient data.

19. The system of claim 13, wherein said patient portal system comprises a patient workflow management module, a supply order module, a user account management module, and an application programming interface.

20. The system of claim 19, wherein said patient workflow management module is configured to provide at least one sample collection form to the patient through said patient portal application, wherein said at least one sample collection form is directly integrated with said patient workflow management module such that the patient's response is reviewable in real-time.
